(19) United States
(12) Patent Application Publication
(10) Pub. No.: US 2025/0099162 A1
(43) Pub. Date: Mar. 27, 2025

(54) CAIMAN RING FORCEPS - MANUAL HF ACTIVATION

(71) Applicant: Aesculap AG, Tuttlingen (DE)

(72) Inventors: Nikolaus Hafner, Tuttlingen (DE); Markus Bauer, Eutingen-Rohrdorf (DE); Nicola Giordano, Villingen-Schwenningen (DE)

(21) Appl. No.: 18/728,882

(22) PCT Filed: Jan. 16, 2023
(86) PCT No.: PCT/EP2023/050842

§ 371 Date: Jul. 14, 2024

(30) Foreign Application Priority Data
Jan. 17, 2022 (DE) 10 2022 100 924.6

(51) Int. Cl. A61B 18/14; A61B 18/00; A61B 18/12

(52) U.S. Cl. CPC A61B 18/1442; A61B 18/1206; A61B 2018/00184; A61B 2018/00601; A61B 2018/0063; A61B 2018/00708; A61B 2018/00922; A61B 2018/0094; A61B 2018/126; A61B 2018/1455

(57) ABSTRACT

A bipolar HF instrument includes a jaws part, a first instrument branch, and a second instrument branch for grasping tissue. The jaws part, first instrument branch and second instrument branch move away from one another during opening and towards one another during closing. A blade actuating element moves a mechanical blade in the jaws part to cut tissue, and/or an HF activation element activates an HF current supply for sealing tissue. The blade actuation element and/or the HF activation element is/are operable simultaneously in each case from two opposite sides of the HF instrument.

### CROSS-REFERENCE TO RELATED APPLICATIONS

This application is the United States national stage entry of International Application No. PCT/EP2023/050842, filed on Jan. 16, 2023, and claims priority to German Application No. 10 2022 100 924.6, filed on Jan. 17, 2022. The contents of International Application No. PCT/EP2023/050842 and German Application No. 10 2022 100 924.6 are incorporated by reference herein in their entireties.

### FIELD

The present disclosure relates to a (medical) bipolar HF instrument preferably of the scissor or pincer type with a first and a second instrument branch (intersecting in a hinge), which form or have a jaw part, for example for grasping tissue, at a distal instrument end portion and which form or have holding/actuating geometries (handles, finger rings, etc.) at a proximal instrument end portion, all of which are provided and configured to move away from each other when opening and toward each other when closing for manual cutting and (HF) sealing of tissue, in particular in open surgery.

### BACKGROUND

In general, surgical sealing and cutting instruments of the above type are known which use a high-frequency (HF) current for sealing via a bipolar technique and a mechanical blade for cutting (biological) tissue. Automatic permanent activation is provided when the branches are pressed together. A blade lock is often integrated into the instrument as a safety feature when the branches are open. Furthermore, an HF button is usually provided to give tactile feedback in two stages (2-click button). The first click indicates to the user that a defined closed state has been reached and the second click activates the HF current. This surgical instrument of the prior art enables fast operation, in particular for experienced users. The surgical instrument has clearly separated functions for cutting and sealing as well as low manufacturing costs.

Alternatively, there is a surgical instrument that uses ultrasound for sealing and cutting, wherein sealing and cutting can be activated manually. Furthermore, this surgical instrument offers the choice between a MIN or a MAX mode on the instrument. It is advantageous that no unintentional activation is possible when closing, in particular since the user has free choice and thus more control and safety.

However, the known surgical instruments have the disadvantage that it is necessary to hold the HF button permanently and this can be unergonomic and tiring for a user in the long term, in particular since several activations per surgery are possible. Another disadvantage can be the variation of the applied pressure. This is because the user can vary the operating pressure on the instrument/its branches with the force of his hand, which may have a negative effect on the sealing result. Other disadvantages include accidental HF activation if compressed too quickly and a preferred orientation during handling due to the asymmetrical arrangement of the blade actuating element.

U.S. Pat. No. 10,188,450,B2 describes pincers having a first and a second shaft (branch), each having a jaw member disposed at a distal end thereof. At least one jaw member is movable from an open position to a closed position in order to grip tissue therebetween. At least one jaw member is configured to reciprocate a blade. A trigger assembly includes a trigger and at least one linkage connected to the trigger and the blade such that rotation of the trigger moves the blade between the retracted position and the extended position. An engagement element movable between a locked position and an unlocked position is also provided. The engagement element is configured to engage the linkage(s) in the locked position to prevent displacement of the blade from the retracted position to the extended position.

U.S. Pat. No. 10,660,694,B2 describes a switch assembly for an electrosurgical instrument with a switch housing, a switch, a first pre-stressing element and an additional pre-stressing element. The switch is arranged within the switch housing and is movable between an activated position for initiating the delivery of electrosurgical energy and a deactivated position for terminating the delivery of electrosurgical energy. The first pre-stressing element is selectively positionable adjacent to and in communication with the switch. The first pre-stressing element includes a first thickness that provides a first resistance to resist movement of the switch between the activated position and the deactivated position when positioned in the switch housing. The additional pre-stressing element is selectively interchangeable with the first pre-stressing element. The additional pre-stressing element has a different thickness that provides a different resistance to resist movement of the switch between the activated position and the deactivated position when positioned in the switch housing.

A bipolar electrosurgical instrument is known from EP 2 671 528 B1 and EP 2 436 330 B1, comprising first and second shafts (branch) each having a jaw member extending from its distal end. Each jaw member is adapted to connect to a source of electrosurgical energy such that the jaw members are capable of selectively conducting energy through tissue held therebetween. A knife channel is configured to move a cutting mechanism back and forth within it. An actuator selectively advances the cutting mechanism. A switch is disposed on the first shaft and configured to be depressed between a first position and at least one subsequent position upon pre-stressing engagement with a mechanical interface disposed on the second shaft. The first position of the switch communicates information to the user corresponding to a desired pressure on the tissue, and the at least one subsequent position is configured to activate the source of electrosurgical energy to deliver electrosurgical energy to the jaw members.

U.S. Pat. Nos. 7,253,667,B2, 9,498,279, B2, US 2019 0356 952 A1, WO 2019 224 634 A1, EP 1 609 430 B1, US 2019 0357 969 A1, WO 2019 224 636 A3, US 2019 0357 967 A1 and WO 2019 224 637 A1 also describe generic electrosurgical instruments. Thereby the above-mentioned WO 2019 224 634 A1 discloses operating options on both sides.

### SUMMARY

The present disclosure is based on the object of providing a bipolar HF instrument which enables simplified operation, in particular over a longer period of time, and furthermore the orientation of the instrument for use is irrelevant. Furthermore, it is in particular an objective of the present disclosure to eliminate or at least improve the above disadvantages of the prior art.

Accordingly, the bipolar HF (hand) instrument has a jaw part (for grasping/retaining/gripping tissue) as well as a first instrument branch and a second instrument branch (for manually operating the jaw part), which are (all) provided and configured to move away from each other when opening and toward each other when closing. In addition, a blade actuating element/blade operating lever or (sliding) button is provided for activating a mechanical blade mounted in the jaw part for cutting tissue, wherein the mechanical blade is provided and configured to be movable from a first position (parking/non-cutting position) to a second position (cutting position) via the blade actuating element. Furthermore, an HF activation element or activation button/knob/switch is provided for activating/releasing an HF power supply, in particular for sealing (coagulating) tissue and, if applicable, a latching device, preferably configured separately, and acting on the branches in order to (selectively) achieve a latched branch state when the instrument branches are closed.

The basic idea of the present disclosure consists in the fact that the blade actuating element and/or the HF activation element are provided and configured in such a way that they can each be operated from two, preferably opposite sides (or sides facing away from each other) of the HF instrument in a further preferably identical or similar manner. This has the advantage that the mechanical blade can always be operated in the same way, regardless of which way round or in which hand the instrument is held. Accordingly, there is no preferred orientation or position of the instrument. This also applies to right-handed or left-handed use. In other words, it is irrelevant how a user holds the bipolar HF instrument, since the operation always functions in the same way due to the double-sided design of the blade actuating element and the HF activation element.

In other words, the present subject matter according to the disclosure is a surgical (hand) instrument of the scissor or pincer type with bipolar HF technology and mechanical (tissue cutting) blade. By opening and closing the instrument branches, the jaw part is also opened and closed. When closed, tissue can be grasped and sealed using HF (high-frequency energy supply) via electrodes arranged in the jaw part. The jaw part can also be used to prepare and manipulate the tissue. When the jaw part is closed, the tissue can also be cut using the blade stored in the jaw part. The actuating devices for separate and individual manual actuation of the HF power supply and the blade are configured and arranged in such a way that essentially the same actuating geometry with essentially the same function/effect on the HF power supply and the blade results on two opposite sides of the instrument or two sides facing away from each other. It is expressly pointed out that the mirroring of the actuating geometry described above is not necessarily only two-dimensional, but may also be three-dimensional. In other words, both actuating devices for the actuation of HF power and blade may, for example, be arranged (mirror image) on the left and right of the instrument in essentially the same way (two-dimensional duplication) or only one actuating device (possibly blade actuation) may be arranged on the left and right of the instrument and the other actuating device (possibly HF power supply) on the top and bottom of the instrument (mirror image in each case) (three-dimensional duplication).

The mechanical blade is movable from a first position to a second position via the blade actuating element. The blade actuating element is moved linearly from its distal resting position in the proximal direction. The mechanical blade moves correspondingly linearly from its proximal resting position in the distal direction and can thus cut tissue gripped in the jaw part.

Furthermore, the instrument preferably has a permanently installed latching device in the form of a pass-through stop. The detent can be used to lock the instrument branches in a defined state (relative position). For engaging, the instrument branches are closed until the latched-in state is reached. For disengaging, the branches are pressed together again slightly until the detent is released. The design of the latching device/detent/pass-through stop is well known from the prior art and therefore does not need to be described further. It has the essential advantage that it can be used to generate a defined closing state in which fluctuations in the closing force are only to be expected to a small extent. This in turn has the advantage that the sealing quality is increased and constant performance is ensured. In addition, the latching device enables the user to hold the tissue securely without triggering HF activation. Another advantage is that the user can relax his hand when the detent is closed. Permanently squeezing of the instrument is not necessary. This makes this disclosure particularly ergonomic.

It is advantageous if the latching device consists of a male part and a female part. In other words, HF can be activated and cut in the closed and latched-in state. As a rule, HF is activated first and sealing is performed. For this purpose, the HF activation element is moved proximally. Preferably, a touch activation is provided so that the user only has to press the button/knob once and does not have to hold it permanently. An HF generator takes over the logic with its integrated algorithm and switches off automatically after successful sealing. However, the user always has the option of stopping the sealing process by pressing the HF activation button/knob again. Cutting can be performed in a second step. To do this, the blade actuating element is pulled proximally. Both the HF activation element and the blade actuating element are automatically reset, preferably by springs.

It is preferable if the sealing and/or cutting functions can be used independently of each other. In other words, the user has the freedom to use the sealing and/or cutting functions independently of each other.

It is preferred if a first rack and pinion gear is provided and arranged in the first instrument branch, in which an input rack is connected to the blade actuating element and an output rack is connected to the mechanical blade, wherein the blade actuating element is linearly movable proximally from a distal resting position. In other words, the deflection mechanism required for this in the first instrument branch is a rack and pinion gear, in which the input rack is attached to the blade actuating element and the output rack is attached to the mechanical blade.

It is advantageous if the input rack and the blade actuating element connected thereto are arranged approximately on the plane of symmetry between the first instrument branch and the second instrument branch.

In other words, the function with the rack and pinion gear represents a significant advantage for the symmetrical use of the instrument. The arrangement of the input rod and the associated blade actuating element approximately on the symmetry plane between the first and second instrument branch has the great advantage that the blade can always be actuated in the same way, regardless of how the instrument is held. There is no preferred orientation or position of the instrument in this respect. This also applies to right-handed or left-handed use.

It is preferred if the bipolar HF instrument also has a blade interlock which is configured to block the actuation of the mechanical blade in an open state and to allow the mechanical blade to move in a defined closed state. Two different safety concepts are provided here, which can be implemented in the bipolar HF instrument.

In a first safety concept, blade actuation is possible as soon as the jaw parts are sufficiently closed, for example when they touch each other. In principle, HF activation/actuation is always possible. This means that there is no locking of the HF activation element. Alternatively, HF can be activated when the instrument is not engaged. This allows the user more application options, but also entails the risk of HF activation with insufficient closing force. In this case, the fixed latching device enables a defined closing state.

In a second safety concept, both blade actuation and HF actuation are only possible when the instrument is in the engaged position of the permanently installed latching device. This ensures that both cutting and HF sealing are only possible in a clearly defined closed state and guarantees maximum safety. However, this option limits the user's options. Experienced users in particular may perceive this safety concept as a severe limitation. It is therefore preferable if the first safety concept is used for experienced users and the second safety concept for less experienced users.

Preferably, the HF activation element is configured and provided in such a way so as to be proximally movable or pivotable or displaceable when performing sealing.

It is advantageous if the HF activation element is configured and provided in such a way that it activates the HF power supply after a first tap activation and switches it off automatically, preferably via an integrated algorithm, after successful sealing, or stops the HF power supply manually after a second tap activation of the HF activation element before successful sealing.

It is preferred if the HF activation element has an inner part which is mounted in a rocking, sliding or pivotable manner on an axis of rotation of the HF activation element in the second instrument branch orthogonal to the proximal-distal direction, wherein the side of the inner part pointing in the direction proximal to the instrument has a contact element with an activation surface which contacts a first HF activation button during actuation of the HF activation element and activates the HF power supply.

In other words, the design and positioning of the HF actuation button also aims to enable fully symmetrical operation of the instrument or to avoid the need for a preferred orientation of the instrument.

According to a first preferred embodiment, the HF activation element is mounted in the second branch so that it seesaws around an axis and can be reached equally well from both sides of the instrument. Furthermore, the positioning of the HF activation element in only one instrument branch has the advantage that the cabling of the HF activation is also only located in one instrument branch. This significantly reduces the effort and costs compared to a concept with actuation buttons/HF activation buttons in both instrument branches.

It is advantageous if the inner part of the HF activation element has a first wing and a second wing, wherein the first wing projects laterally from the second instrument branch and the second wing protrudes opposite the first wing on the other side of the second instrument branch.

In other words, the HF activation element consists of several parts, an inner part and a first and a second wing. Alternatively, a one-piece design is also conceivable under other assembly conditions.

Alternatively, it is conceivable to design the HF activation element as a slider that is linearly movable in the proximal and distal directions. For this purpose, it is advantageous if the HF activation element is movably guided in a slit in a housing of the second instrument branch, for example. This has the advantage that exactly one electronic HF activation button is required.

The activation surface of the HF activation element according to the above embodiment is provided to press the first (electronic) HF activation button.

Preferably, the contact element is provided and configured cam-shaped toward the first HF activation button, or a second rack and pinion gear is placed distal to the first HF activation button so as to contact it with the activation surface during actuation of the HF activation element.

In other words, an alternative embodiment with exactly one electronic HF activation button is possible. For this purpose, the inner part of the HF activation element has a type of cam or the like, which comes into engagement with the first HF activation button during rotation/pivoting about the axis of rotation of the HF activation element. An additional return spring is provided and may be necessary.

An alternative embodiment using (exactly) a first HF activation button is provided with a rack and pinion gear. In this case, the inner part of the HF activation element is also rotatable and connected to a pinion. Two racks are placed on two sides of the pinion, each of which can transmit its movement to a contact element/activation element/pressure plate. The contact element then actuates the first HF activation button. Depending on the design of the HF activation button, the contact element is superfluous and the racks can press directly on the HF activation button. In order to ensure reliable functioning, it is advantageous if the racks are fitted with return springs.

It is advantageous if a second HF activation button is provided and arranged next to the first HF activation button parallel to the inner part of the HF activation element. In other words, the activation surface of the contact element exerts a force on one of the two HF activation buttons by rotating/pivoting/displacing the HF activation element, thereby triggering activation of the HF process. The HF process can also be stopped manually if required by rotating/pivoting/displacing the HF activation element again. In addition to the fully symmetrical design, the above embodiment has the advantage that the HF activation element is/can be actuated by pulling as well as pushing. This opens up several ergonomic operating options for the user.

It is preferred if returning to a non-actuated resting position is provided via an integrated spring in the HF activation buttons. Alternatively, an additional return spring may be provided in the form of a sheet metal/leaf spring, a torsion spring or something similar.

It is therefore advantageous if the inner part of the HF activation element is supported by a spring element, for example a compression spring. This has the advantage that the spring element causes the entire HF activation element to protrude a little in the axial direction of its axis of rotation. As a result, the activation surface, which would press on the first and/or second HF activation button, is disengaged and no HF power supply can be activated when the HF activation element is operating in this position. In a closed state, the HF activation element is pressed in the second instrument branch. In this position, the activation surface is brought back into engagement with the first and/or second HF activation button by closing the first and second instrument branches and the resulting compression of the compression spring in the second instrument branch. It is advantageous if the HF activation element has a separate return spring that permanently forces it into a rest position in which the wings of the HF activation element are perpendicular to the main plane of the instrument. The return spring may be a leaf spring, tension spring, torsion spring or something similar.

It is advantageous if a commercially available electronic button can be used for the first HF activation button and/or the second HF activation button.

It is preferred if the first HF activation button and/or the second HF activation button is/are arranged in a proximal direction away from the inner part of the HF activation element but facing it on a printed circuit board in the second instrument branch. In other words, it is preferred that the first HF activation button and/or the second HF activation button are located on a printed circuit board on which they are interconnected in an OR logic. This ensures that an action takes place no matter which one of the HF activation buttons is pressed.

Alternatively, it is preferable if the HF activation element is designed as a lever. In this case, it is provided that the position of such a lever can be switched as soon as the bipolar HF instrument is rotated, so that fully symmetrical actuation of the instrument is also possible. The lever is therefore located on both sides of the instrument or of the second instrument branch. In this respect, it is advantageous if a first and a second HF activation button are provided/placed in the instrument branch or in the housing of the instrument branch, which are opposite each other in such a way that when the lever is in a first position, the first HF activation button is pressed during actuation, and when the lever is in position 2, the second HF activation button is pressed during actuation. Switching from position 1 to position 2 is preferably realized by a snap mechanism so that the lever can be noticeably configured to engage in the defined positions.

It is preferable if, according to the second safety concept, the installation of a locking device of an HF activation element is provided. In other words, a locking element prevents actuation of the HF activation element in the open state. In a closed state, actuation is enabled.

It is advantageous if the locking element has a plunger that protrudes from a handle shell of the second instrument branch and can be brought into contact with an opposite handle shell of the second instrument branch when the instrument is closed. A locking element, for example a pin, is provided next to the plunger. The pin is provided and configured to interact with a bore in the inner part of the HF activation element. In an open state, the pin is located in the bore and rotation/actuation of the HF activation element is not possible. In a closed state, the pin is pushed out of the bore by the plunger and rotating/operating of the HF activation element is possible. Here it is preferable if the locking element is equipped/configured with guiding elements on two sides in order to prevent the locking element from rotating.

It is preferred if the blade actuating element has a different shape to the HF activation element. Preferably, the blade actuating element has a round configuration and the HF activation element has a different shape, e.g. a rectangular shape. Furthermore, it is additionally or alternatively preferred if the HF activation element has a different color, preferably blue, than the rest of the instrument, in particular since blue is usually associated with HF activation in surgery. Furthermore, it is advantageous if the HF activation element is additionally or alternatively provided at a sufficient distance from the blade actuating element. These differences have the advantage that the risk of confusion between the blade actuating element and the HF activation element can be reduced as far as possible.

It is preferred if the distance between the blade actuating element and a handle of the first instrument branch is 76±20 mm and the distance between the HF activation element and the handle of the first instrument branch is 42±20 mm.

It is advantageous if the HF activation element is sealed against penetrating liquids, in particular blood, water, saline solution, etc. It is preferred if the first and second handle shells of the first and/or second instrument branch are provided with a circumferential seal. Alternatively or additionally, it is provided if a seal is only present in the area of the HF activation element. In other words, due to the use of the instrument in open surgery, another important aspect for the HF activation element is sealing against penetrating liquids. It therefore has to be ensured that the ingress of liquid does not lead to unwanted HF activation. For this purpose, it is provided that the HF activation element is sealed accordingly. Sealing the handle shells with seals, for example TPE, is preferred. The seals may run around the entire contour of the handle shells and/or may only be present in the area of the HF activation element. The seal may be molded into a handle shell or may be a separate part that is inserted during assembly. The sealing effect is achieved by pressing or screwing it to the second handle shell.

Alternatively or additionally, it is preferred if the first and/or second HF activation button used is already configured to be sealed ex works. In other words, the use of sealed HF activation buttons that are already sealed ex works is provided. In this case, the printed circuit board on which the HF activation buttons are mounted/soldered also have to be sealed to prevent a short circuit at the terminals of the printed circuit board. For this purpose, it is preferable if the printed circuit board is encapsulated with a plastic material, for example.

Alternatively or additionally, it is preferred if an elastomer sealing ring (O-ring) is provided on an axis/shaft of the inner part of the HF activation element.

Alternatively, the present disclosure relates to a bipolar HF instrument which can be used alternatively. The bipolar HF instrument is provided with a jaw part with a first instrument branch and a second instrument branch for grasping tissue, which are provided and configured to move away from each other when opening and toward each other when closing, a blade actuating element for activating a mechanical blade in the jaw part for cutting tissue, which is provided and configured to be movable from a first position to a second position via the blade actuating element, exactly two HF activation buttons for activating an HF power supply for sealing tissue, and a latching device, preferably additionally connectible, for achieving a latched-in state with the instrument branches closed, wherein the blade actuating element and the HF activation element are provided and configured symmetrically so as to be equally operable from both sides of the HF instrument, wherein one HF activation element each is mounted laterally on the first and the second instrument branch.

In other words, the HF activation element may be placed laterally on the first and second instrument branches to improve ergonomics. This means that the instrument is also fully symmetrical. This embodiment preferably has an additionally connectible latching device, which can be additionally connected on rotation with the aid of a proximally mounted rotary lever. In order to ensure that the user applies sufficient closing force when the latching device is switched off, an additional control device, here in the form of a third button, is provided. The third button is connected in an AND logic with the HF activation buttons and has to be permanently activated so that the HF power supply can be activated. Further control devices are provided as alternative or additional sensors, such as pressure sensors, distance sensors or something similar. In this way, the user is given as many application options as possible.

### BRIEF DESCRIPTION OF THE DRAWINGS

FIG. 1 shows a bipolar HF instrument in an open and disengaged state.

FIG. 2 shows the bipolar HF instrument in a closed and non-latched-in state.

FIG. 3 shows the bipolar HF instrument in closed and latched-in state.

FIG. 4 shows the bipolar HF instrument with open instrument branches.

FIG. 5 shows the bipolar HF instrument with open instrument branches and the blade, wherein a part of the first instrument branch and the jaw part are hidden.

FIG. 6 shows an HF activation element according to a first embodiment.

FIG. 7 shows a top view of an HF activation element with additionally shown electronic buttons and printed circuit board according to the first embodiment.

FIG. 8 shows an HF activation element according to a second embodiment.

FIG. 9 shows an HF activation element using a rack and pinion gear according to a third embodiment.

FIG. 10 shows an HF activation element as a slider according to a fourth embodiment.

FIGS. 11A and 11B show the HF instrument with a lever as HF activation element.

FIG. 12 shows the HF instrument from above.

FIG. 13 shows the arrangement of the HF activation button and the lever as an HF activation knob.

FIGS. 14 and 15 show the bipolar HF instrument with an HF activation element according to a second safety concept.

FIGS. 16 and 17 show the HF instrument with a locking element of the HF activation element.

FIGS. 18, 19 and 20 show the locking element of the activation element.

FIG. 21 shows the inner part of the HF activation element.

FIG. 22 shows the distances between the individual components of the HF instrument.

FIG. 23 shows the HF activation element with a seal of the HF activation element.

FIG. 24 shows an HF activation button.

FIG. 25 shows the inner part of an HF activation element with sealing ring.

FIGS. 26 and 27 each show a further alternative embodiment of the HF instrument.

FIG. 28 shows a plan view of the HF instrument according to FIG. 1-FIG. 5.

### DETAILED DESCRIPTION

Configuration examples of the present disclosure are described below on the basis of the associated Figures.

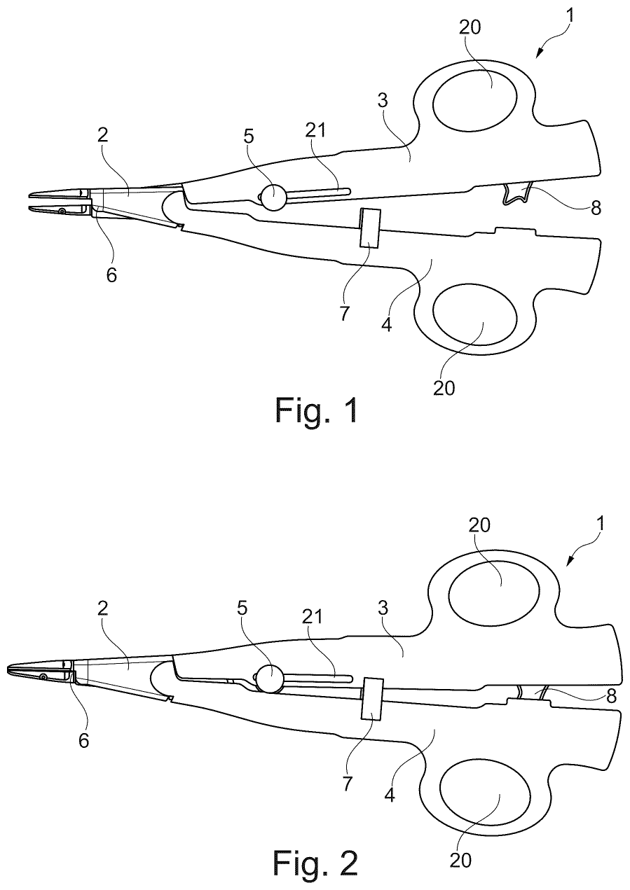

FIG. 1 shows a bipolar HF instrument 1 of the scissor or pincer type in the open and disengaged state. The bipolar HF instrument 1 has a jaw part 2, a first instrument branch 3 and a second instrument branch 4, which are coupled to the jaw part via a gear or in one piece. When the first instrument branch 3 and the second instrument branch 4 are opened and closed, they pivot about a hinge not shown further in FIG. 1, as a result of which the jaw part 2 also opens and closes accordingly. In the closed state, the jaw part 2 can grasp/grip/hold tissue and can seal it at electrodes not shown further in jaw part 2 via an HF power supply. This HF technology is well known from the prior art, for example according to the present applicant.

Furthermore, the first instrument branch 3 and the second instrument branch 4 each have an annular instrument actuating lug 20 in the proximal half of the instrument branches 3 and 4, into which fingers of an actuating hand can be inserted. Other instrument actuating forms are of course also conceivable, such as open brackets or reach-through slots in the instrument branches 3, 4.

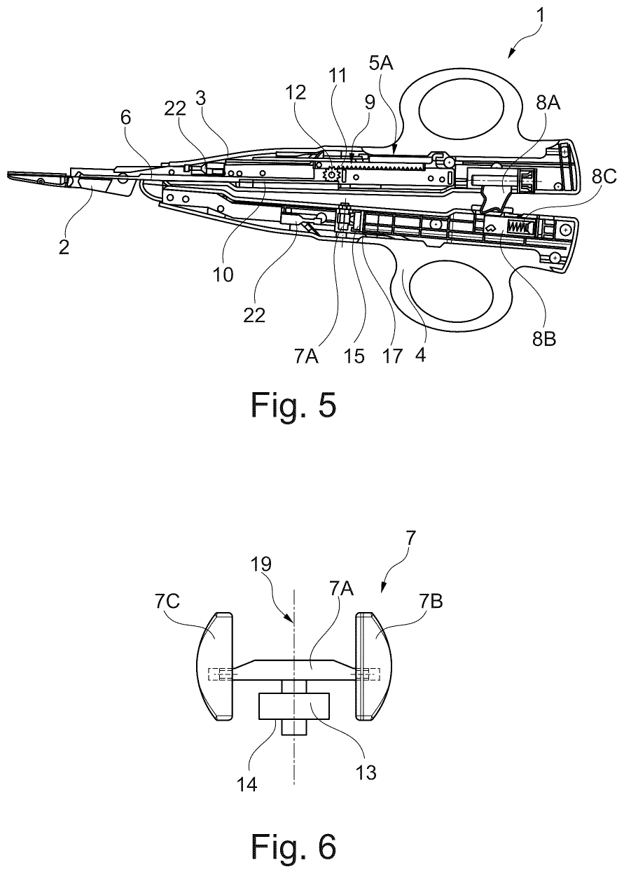

A mechanical blade 6 is provided in the instrument 1, in particular in the area of the jaw part 2, which is preferably movable in the longitudinal direction of the instrument via a blade actuating element/button/knob 5. The mechanical blade 6 is provided to cut the tissue held in the jaw part 2. The arrangement of the mechanical blade 6 is shown in more detail in FIG. 5.

The blade actuating element 5 is arranged in the first instrument branch 3. The blade actuating element 5 is configured to be moved linearly from its distal resting position proximally (i.e. in the direction away from the jaw part 2/away from the patient). According to FIG. 1, the blade actuating element 5 is mounted within a slit 21 to be longitudinally displaceable and which is formed in an instrument branch 3. The blade actuating element 5 is arranged between the jaw part 2 and the annular instrument actuating lug 20 of the first instrument branch 3 on the side adjacent to the second instrument branch 4. The blade actuating element 5 is preferably configured according to FIG. 1 as a pin projecting laterally from the instrument branch 3.

The jaw part 2 of the bipolar HF instrument 1 furthermore has HF litz wires (not shown), which are supplied with HF energy after actuation of an HF activation element 7 and transmit this energy to the electrodes in the jaw part 2. The HF litz wires are connected to the flat connector 22. One HF litz wire is connected to each element of the jaw part 2. The elements of the jaw parts 2 are electrically insulated from each other (insulation not shown).

The HF activation element 7 is arranged in/on the second instrument branch 4 on the side adjacent to the first instrument branch 3. Here, the HF activation element 7 is positioned closer to the ring-shaped instrument actuating lug 20 than the blade actuating element 5 and its associated slit 21 in the first instrument branch 3. The HF activation element 7 is preferably configured as a rectangular tip button as shown in FIG. 1.

Finally, the bipolar HF instrument 1 has a latching device 8 of generally known design at the level of the ring-shaped instrument actuating lugs 20. The latching device 8 has a male part 8A and a female part 8B, which are shown in more detail in FIG. 4. The latching device 8 is provided on the sides of the instrument branches 3 and 4 facing each other. In FIG. 1, the latching device 8 is shown in a disengaged state.

FIG. 2 shows the bipolar HF instrument 1 in the closed and not (yet) latched-in state of the latching device 8. In FIG. 2, the HF instrument 1 is shown according to FIG. 1, with the first instrument branch 3 and the second instrument branch 4 still closed. However, the jaw part 2 is already closed, although the closing force has not yet reached a certain value. In the closed and non-latched-in state shown in FIG. 2, however, depending on which safety concept is used, it may already be possible to cut and activate HF.

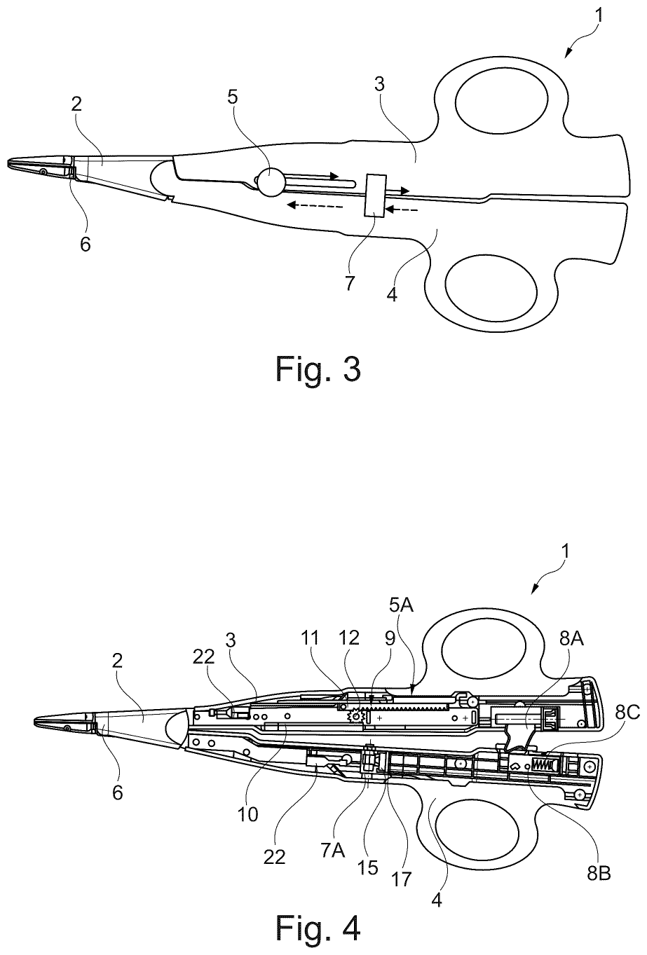

When the instrument branches 3 and 4 are closed further, the latching device 8 engages fully. FIG. 3 shows the bipolar HF instrument 1 in such a closed and latched-in state. FIG. 3 shows the HF instrument 1 as shown in FIG. 1 and FIG. 2, with the first instrument branch 3 and the second instrument branch 4 fully closed. The latching device 8 is latched in. In the latched-in state, the blade actuating element 5 and the HF activation element 7 lie almost in the middle between the two instrument branches 3 and 4 (i.e. essentially on the symmetry plane of the instrument 1).

In FIG. 3, a solid arrow is shown pointing proximally from the blade actuating element 5. This arrow represents the direction of movement of the blade actuating element 5 in order to move the mechanical blade 6 for cutting. A dashed arrow pointing distally, parallel to the solid arrow, indicates the direction of the reset path of the blade actuating element 5 in order to return the blade 6 to its rest position.

FIG. 3 also shows a solid, short arrow pointing proximally from the HF activation element 7. This arrow represents the direction of movement of the HF activation element 7 in order to activate the HF power supply. A dashed arrow pointing distally parallel to the short solid arrow indicates the direction of the reset path of the HF activation element 7.

FIG. 4 shows the bipolar HF instrument 1 with open instrument branches 3 and 4 in longitudinal section. The first instrument branch 3 and the second instrument branch 4 each consist of two half-handle shells, each of which encloses a cavity, whereby the inner workings of the first and second instrument branches 3 and 4 can be seen in FIG. 4.

FIG. 4 shows the handle shell of the first instrument branch 3 with a first rack and pinion gear 9 with an input rack 10 and an output rack 11. The input rack 10 is firmly connected to the blade actuating element 5 (not shown in FIG. 4). The output rack 11 is firmly connected to the mechanical blade 6 (not shown in FIG. 4). The input rack 10 and the output rack 11 are connected to each other via an interposed pinion 12 in such a way that when the blade actuating element 5 is actuated by a user in the proximal direction, the mechanical blade 6 is moved from its proximal resting position to the distal direction in order to cut the tissue held in the jaw part 2. The rack and pinion gear 9 serves here as a deflection mechanism in the first instrument branch 3. A return spring 5A is also provided for the aforementioned blade mechanism in such a way that the blade actuating element 5 and thus the blade 6 are automatically pushed back into their respective resting position.

FIG. 4 shows the handle shell of the second instrument branch 4 with the inner life of the HF activation element 7. In detail, an inner part 7A of the HF activation element 7 is indicated, which is arranged distal to a first HF activation button 15. If a second HF activation button 16 is provided, it is arranged to the side of the first HF activation button 15 shown and is therefore not visible in FIG. 4. The HF activation button 15 shown is arranged on a printed circuit board 17. If a first HF activation button 15 and a second HF activation button 16 are provided, they are interconnected via the printed circuit board 17 via an OR logic. A flat connector 22 is also provided in both the first instrument branch 3 and the second instrument branch 4 in order to contact the HF litz wires of the jaw part 2.

FIG. 4 furthermore shows the latching device 8 in longitudinal section. The latching device 8 has a male part 8A on the side of the first instrument branch 3 and a female part 8B on the side of the second instrument branch 4. When pressed together, the two parts 8A and 8B latch into each other. The latched-in state can be released again by pressing them together again using a built-in return spring 8C.

FIG. 5 shows the bipolar HF instrument 1 with open instrument branches 3 and 4 and the blade 6, wherein a part of the first instrument branch 3 and the jaw part 2 are hidden. FIG. 5 shows the same components as already explained for FIG. 4. In addition, the mechanical blade 6 is shown, which is arranged in the jaw part 2 and in the first instrument branch 3 and connected to the output rack 10.

FIG. 6 shows an HF activation element 7 according to a first embodiment. The HF activation element 7 has an inner part in the form of a crossbar 7A, which connects a first actuating wing or knob 7B and a second actuating wing or knob 7C to each other. According to this embodiment, one end of the crossbar-shaped inner part 7A is glued and/or pressed into/onto the first actuating wing 7B or the second actuating wing 7C. The first actuating wing 7B and the second actuating wing 7C are arranged outside and laterally, i.e. on the sides of the second instrument branch 4 that are opposite or facing away from each other. The inner part 7A is located in the interior of the second instrument branch 4. A contact element 13 with an activation surface 14 is attached to a central section of the inner part 7A in the proximal direction of the HF instrument or, in FIG. 4, in the downward direction. When the user actuates the HF activation element 7, the contact element 13 connected to the inner part 7A presses the activation surface 14 onto the HF activation button 15 (not shown in FIG. 6) and the HF power supply is activated.

The contact element 13 is configured and mounted on a central axis 19 of the HF activation element 7, i.e. centrally on the inner part 7A in a T-shape relative to the latter, as already indicated above. The activation surface 14 is arranged on the side of the contact element 13 facing the HF activation button 15. The HF activation element 7 is mounted in the second instrument branch 4 in a rocking manner along the central axis 19 in such a way that the two actuating wings 7B and 7C project laterally from the second instrument branch and can thus be reached and actuated equally well from both sides of the instrument 1.

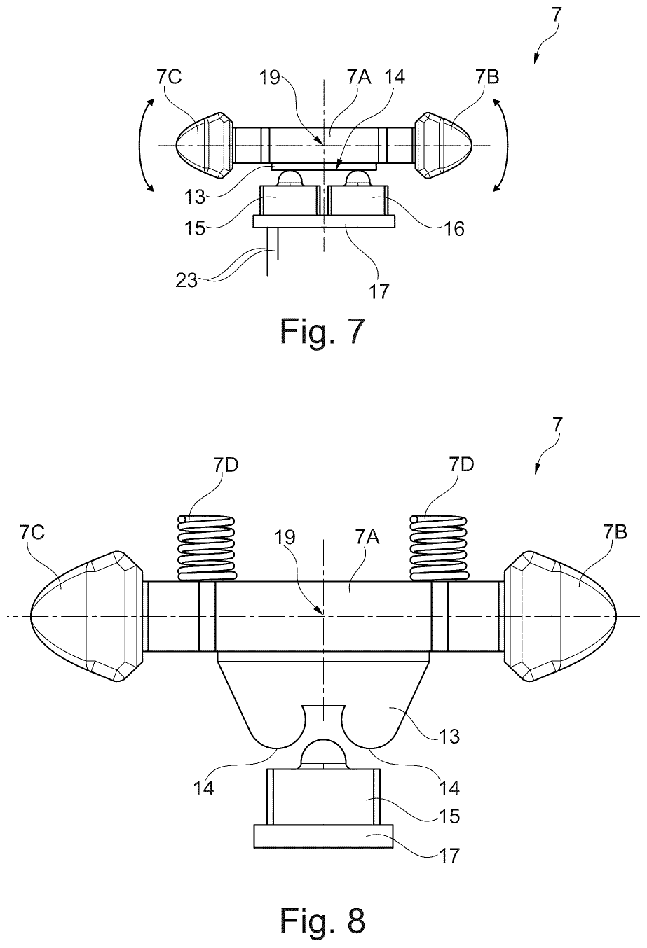

FIG. 7 shows the HF activation element 7 according to the first embodiment. According to FIG. 7, a crossbar-shaped inner part 7A of the HF activation element 7 with a first actuating wing 7B and a second actuating wing 7C is shown. A contact element 13 with the activation surface 14 is attached to or configured on the inner part 7A in the center of the inner part 7A of the HF activation element 7 in the direction of the first HF activation button 15 and a second HF activation button 16.

In this embodiment, the first second HF activation buttons 15, 16 are provided, which are arranged next to each other and, in FIG. 7, below the activation surface 14, i.e. proximal to the activation surface 14 as shown in FIG. 5. Both HF activation buttons 15, 16 are arranged on a (single) printed circuit board 17. Two terminals 23 are provided on the printed circuit board 17, which are used for the power supply. By rotating/pivoting/seesawing the HF activation element 7 in the longitudinal direction of the central axis 19, irrespective of which actuating wing 7B, 7C on the opposite side of the instrument 1 the user is using, the activation surface 14 presses on one of the two HF activation buttons 15, 16, which releases the HF current to the electrodes in the jaw part 2.

FIG. 8 shows an HF activation element 7 according to a second embodiment. The second embodiment corresponds almost exactly to the first embodiment, with the difference that the contact element 13 is formed with two cam-shaped projections arranged next to each other, which surround the HF activation button 15 on the outside (without contact), so that the contact element 13 or its projections come into contact with the HF activation button 15 during actuation of the HF activation element 7 with a corresponding seesawing movement of the crossbar-shaped inner part 7A. Due to the double-cam-shaped contact element 13, exactly one electronic HF activation button 15 is sufficient and contact of the activation surface 14 on the side facing the HF activation button 15 with the HF activation button 15 is ensured.

On the distal side of the inner part 7A opposite the contact element 13 (in all embodiments of the HF activation element 7 described above), two return springs 7D are provided, spaced apart in the transverse direction of the instrument, which are supported on the second instrument branch 4 on the one hand and on the crossbar-shaped inner part 7A on the other hand, in order to push the HF activation element 7 back into the (contactless) initial position, regardless of which side of the instrument 1 is actuated.

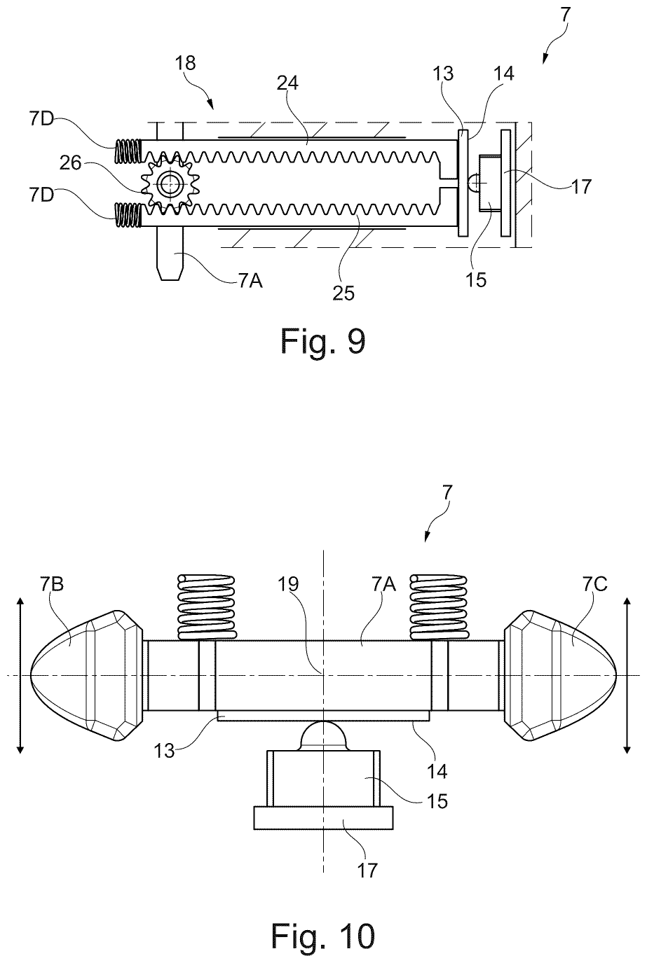

FIG. 9 shows an HF activation element 7 with a second rack and pinion gear 18 according to a third embodiment. The second rack and pinion gear 18 has a first rack 24 and a second rack 25, which lie opposite each other and are operatively connected to each other at distal end portions via a pinion 26. At the distal ends, at which the pinion 26 is located, a return spring 7D acting in the proximal direction is attached to both the first rack 24 and the second rack 25. At the other, proximal ends of the first rack 24 and the second rack 25, a contact element 13 with an activation surface 14 is provided in the direction of an HF activation button 15 arranged proximally to the HF activation element 7. The HF activation button 15 is arranged on a printed circuit board 17. This means, the protruding rack and pinion gear 18 is provided in the second instrument branch 4 in such a way that the HF activation button 15 is arranged in a direction proximal to the rack and pinion gear 18. Finally, an operating element 7A in the form of a crossbar is shown, which protrudes from one side of the second instrument branch and which is connected to the pinion. A further operating element of the same design (not shown further) is also connected to the pinion and projects from the second instrument branch on the opposite side of the latter.

If the operating element 7A shown is now moved in a proximal direction, one rack 25 also moves in a proximal direction, while the other rack 24 is moved in a distal direction. As a result, the contact element 13 undergoes a tilting movement similar to the embodiments described above, whereby the HF activation button 15 is actuated. When the operating element 7A is released, the pre-tensioning springs 7D cause the racks 24, 25 to return to their initial position. In principle, however, it is also possible to couple operating elements with the axis of rotation of the pinion 26 in order to trigger the counter-rotating longitudinal movements of the racks 24, 25 via the manually actuated rotation of the pinion 26.

FIG. 10 shows an HF activation element 7 as a slider according to a fourth embodiment. The fourth embodiment is configured according to FIG. 8, wherein the contact element 13 with the activation surface 14 is configured flat instead of cam-shaped. Furthermore, in this case the crossbar-shaped inner part 7A is not rocker-shaped as in the embodiment according to FIG. 8, but longitudinally displaceable in the second instrument branch 4.

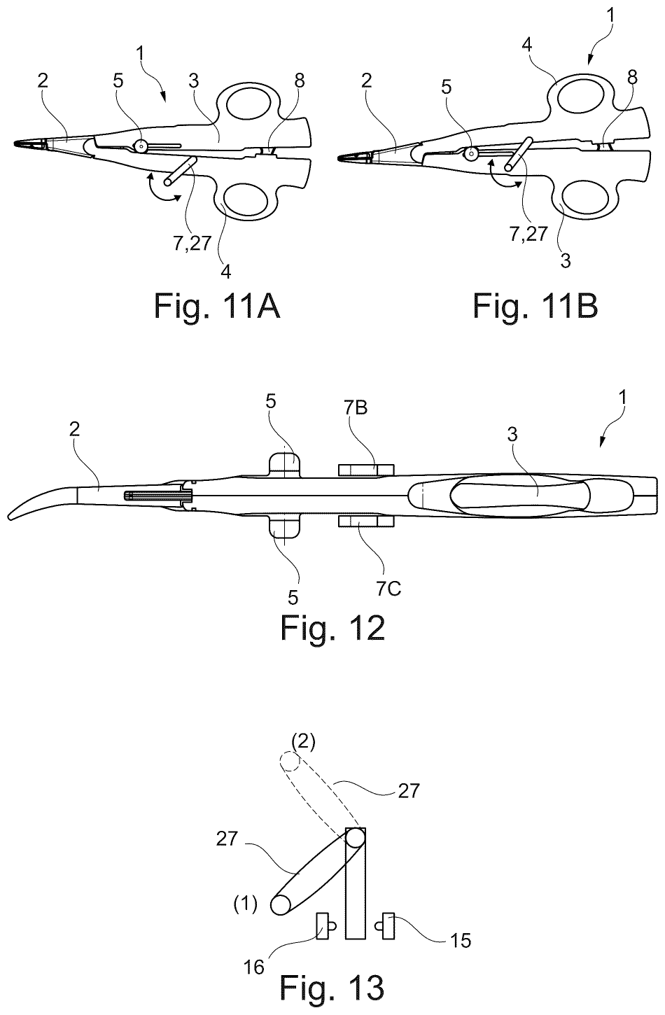

FIGS. 11A and 11B and 12 show the HF instrument 1 with a lever 27 as HF activation element 7 on the opposite sides of the instrument. According to FIG. 11A, this sixth embodiment of the HF activation element 7 shows the one lever 27, which is pivotally guided laterally from the second instrument branch 4 parallel thereto. In FIG. 11A, the HF instrument 1 is aligned in accordance with the previous Figures. In FIG. 11B, on the other hand, the orientation of the HF instrument 1 is reversed, i.e. the first instrument branch 3 is shown pointing downward and the second instrument branch 4 is shown pointing upward, so that both levers 27 can be shown on opposite sides of the instrument 1. Due to the almost central arrangement of the HF activation element 7 on the side of the second instrument branch 4 closest to the first instrument branch 3, the operation of the lever 27 is the same regardless of the orientation of the HF instrument 1.

FIG. 13 shows the arrangement of two HF activation buttons 15 and 16 and the lever 27 as HF activation element 7 according to the sixth embodiment in a schematic diagram. According to FIG. 13, the levers 27 are non-rotatably connected to a contact rod, which extends along the second instrument branch 4, preferably in a proximal direction. Furthermore, a first HF activation button 15 and a second HF activation button 16 are provided, which are arranged opposite each other in such a way so as to initially receive the contact rod between them without contact. If the one lever 27, shown in full line, is now in a first position (1) in which the contact rod has no contact with the HF activation buttons 15 and 16 and is actuated counterclockwise from this position, then the first activation button 15 is pressed by the contact rod. In the event that the instrument 1 is rotated, as shown in FIGS. 11A and 11B, the lever 27 shown in dashed lines in its second position (2) is in the same spatial pose as the lever 27 shown in solid line in FIG. 13, wherein in this case the two HF activation buttons 15 and 16 consequently come to lie above the lever 27. If this lever 27, shown as a dashed line, is now actuated counterclockwise, the contact rod comes into contact with the second HF activation button 16 and this is consequently pressed.

It is also possible for the two levers 27 to swap their respective positions shown. In this case, switching from position (1) to position (2) is preferably realized by a snap mechanism between lever 27 and contact rod so that the lever noticeably engages in the defined positions (not shown).

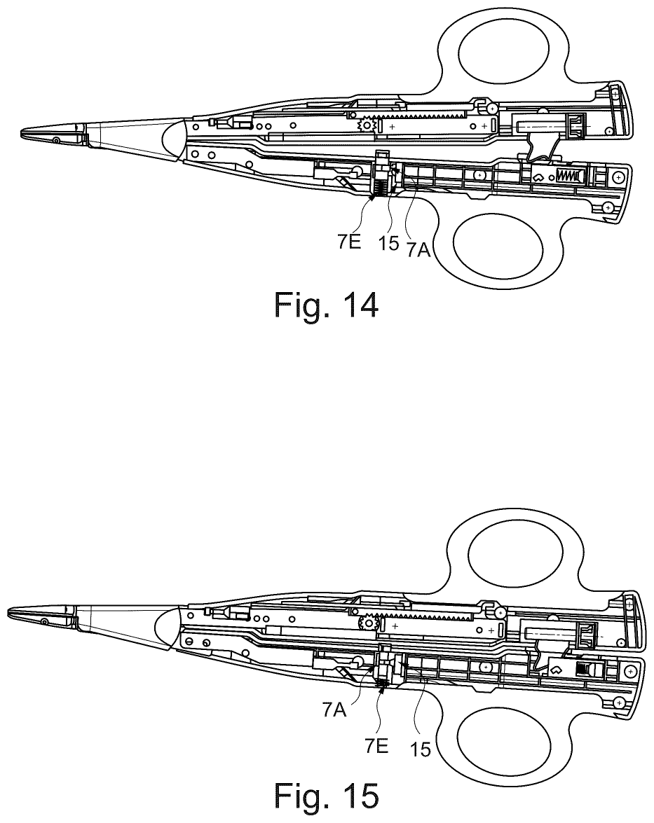

FIGS. 14 and 15 show the bipolar HF instrument 1 with an HF activation element 7 according to a defined safety concept. In FIG. 14, the HF instrument 1 is closed and not engaged. In FIG. 15, the HF instrument 1 is closed and engaged.

The inner part 7A of the HF activation element 7 is supported by a spring element 7E, preferably a compression spring. The spring element 7E is arranged in such a way that the entire HF activation element 7 protrudes a little from the instrument branch 4 in the axial direction of its seesaw axle 19 or is displaced in the axial direction of its seesaw axle 19. As shown in FIG. 14, this means that the activation surface 14, which is intended to press on the first HF activation button 15, can no longer be brought into operative engagement with the HF activation button 15. When actuating the HF activation element 7 in this displaced position, no HF energy can be activated.

As soon as the HF instrument 1 is in a closed and latched-in state, the HF activation element 7 is pushed back again by the first instrument branch 3 against the preload force of the spring element 7E, so that the activation surface 14 can come into engagement with the first and/or second HF activation button 15 and/or 16. This safety concept therefore basically provides for a spring-loaded displacement of the HF activation element 7 into a non-functional position, which is only released again when the two instrument branches 3, 4 reach a fully closed position by the other, first instrument branch 3 pushing the HF activation element 7 back into its functional position.

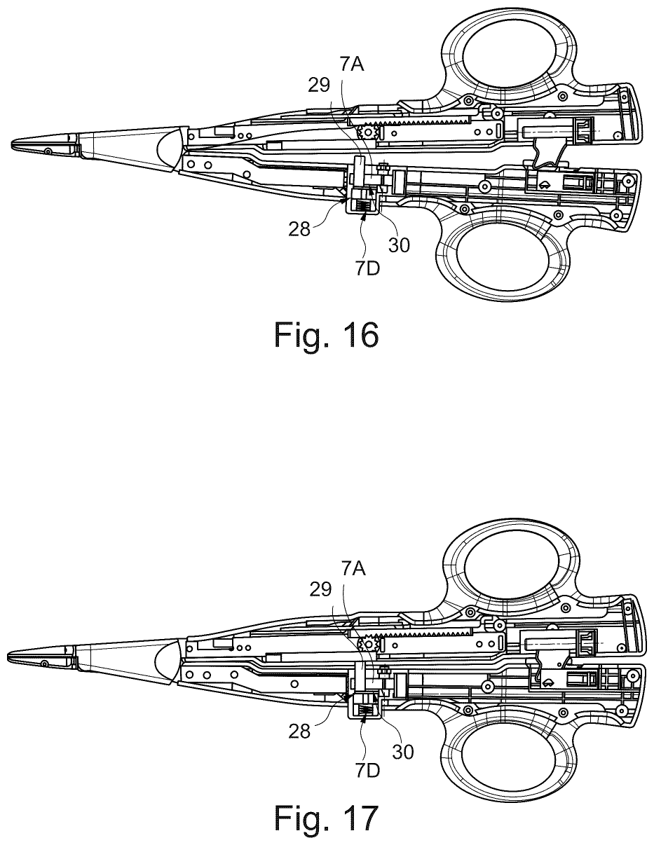

FIGS. 16 and 17 show the HF instrument 1 with a locking element of the HF activation element 28 as another safety concept. In this case, a locking element of the HF activation element 28 is installed/integrated in the HF instrument 1. As a result, the user is enabled to actuate in a closed and latched-in state, provided that he triggers the locking element of the HF activation element 28 accordingly.

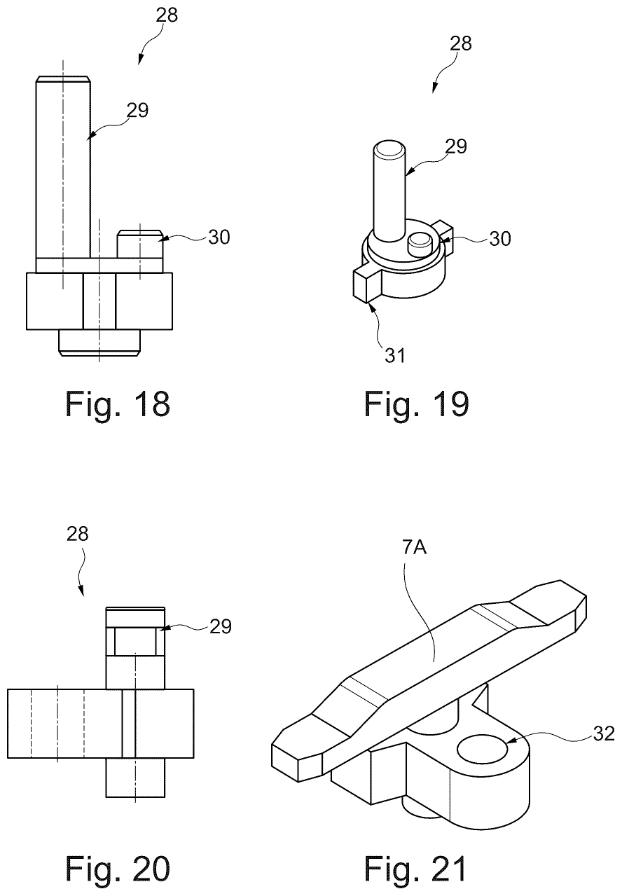

The locking element 28 of the activation element according to FIGS. 18 and 19 has a plunger 29, which protrudes from the handle shell of the second instrument branch 4 and can be brought into contact with the opposite handle shell of the first instrument branch 3 when the HF instrument 1 is closed. A pin 30 is located next to the plunger 29. The pin 30 interacts with a bore 32 as shown in FIG. 21 in the inner part 7A of the HF activation element 7. In an open state of the HF instrument 1 as shown in FIG. 16, the pin 30 is located in the bore 32 and rotating/seesawing/actuating of the HF activation element 7 is not possible. In a closed state of the HF instrument 1 as shown in FIG. 17, the pin 30 is pushed out of the bore 32 by the plunger 29 and the rotating/seesawing/actuating of the HF activation element 7 is possible.

In order to prevent the locking element 28 from rotating, guiding elements 31 in the form of sliding tabs as shown in FIG. 19 are configured on two diametrically opposite sides of the locking element 28, which are slide-mounted in corresponding guide grooves in the second instrument branch 4. This ensures that the pin 30 can retract into the bore 32 when the instrument is opened.

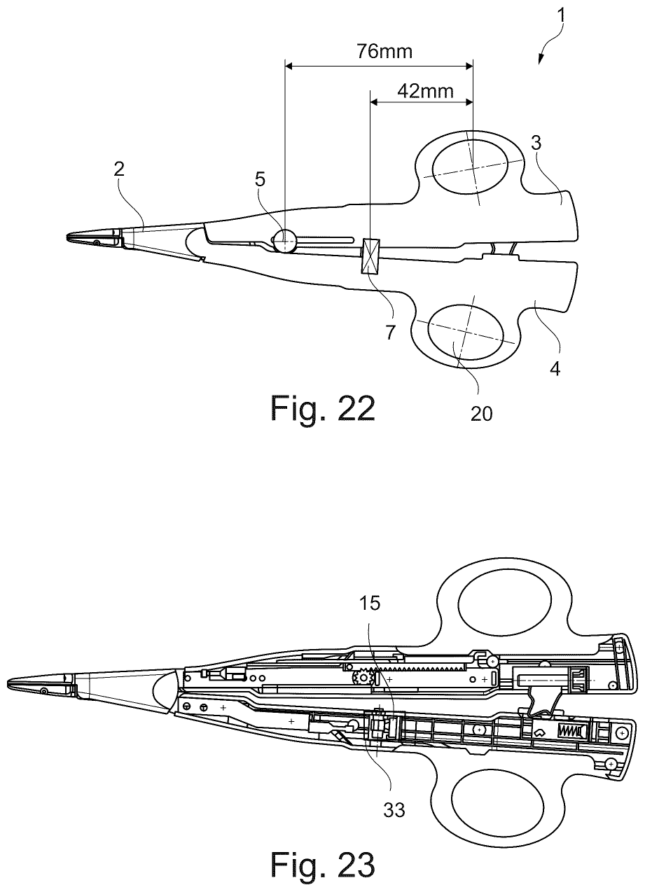

FIG. 22 shows the distances between the individual components of the HF instrument 1. In order to avoid confusion between the HF activation element 7 and the blade actuating element 5 during use, the HF activation element 7 and the blade actuating element 5 are placed at a sufficient distance from each other. It is preferred if the distance from the center of the instrument actuating lug 20 to the center of the blade actuating element 5 is preferably between 56 and 96 mm, more preferably between 66 and 86 mm and more preferably 76 mm. In addition, it is preferred if the distance from the center of the instrument actuating lug 20 to the center of the HF activation element 7 is preferably between 22 and 62 mm, more preferably between 32 and 52 mm and more preferably 42 mm.

FIG. 23 shows the HF activation element 7 with a seal 33. FIG. 23 shows the HF instrument 1 according to the preceding descriptions, wherein the HF activation element 7 and the first and/or second HF activation button 15 and/or 16 are sealed with a TPE seal 33.

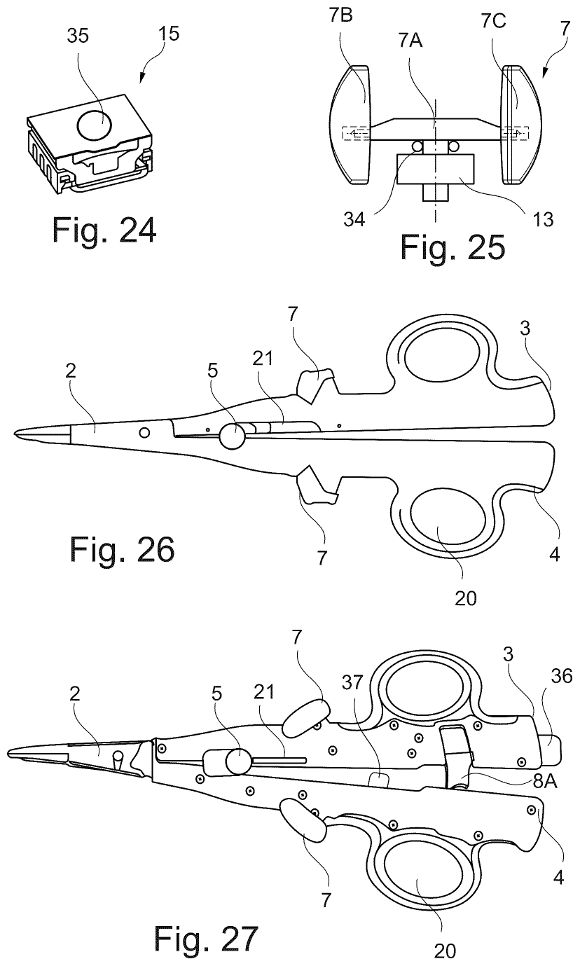

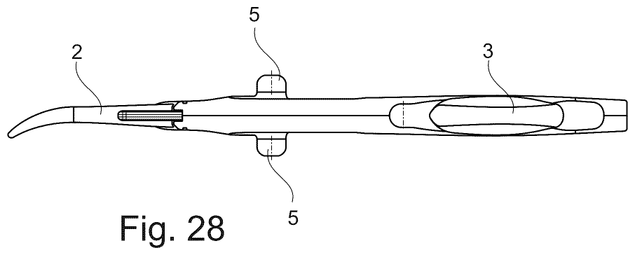

FIG. 24 shows an HF activation button 15 or 16, wherein the HF activation button 15 or 16 is already sealed or encapsulated ex works via a casing, which has a concave, preferably hemispherical protrusion in the contact area of the HF activation button. When the protruding hemisphere 35, on which the activation surface 14 presses during actuation of the HF activation element 7, is touched, the HF energy is supplied.

FIG. 25 shows the HF activation element 7 according to FIG. 6 with a sealing ring 34. The sealing ring 34 is fitted around the T-shaped contact element 13 on the side facing the inner part 7A of the HF activation element 7, so that the crossbar-shaped inner part 7A and the contact element 13 together with the contact surface 14 can be separated from each other in a fluid-tight manner. The sealing ring 34 is preferably an elastomer sealing ring/O-ring.

FIGS. 26 and 27 show a further alternative embodiment of the HF instrument 1. Here, the HF instrument 1 is configured in accordance with the above Figures, with the HF activation element 7 being present in duplicate. In this case, the two HF activation buttons 7 are located on the top of the respective instrument branches 3 and 4, i.e. on the top and bottom of the instrument, whereas the blade actuating element 5 remains on the left and right sides of the instrument in accordance with the previously described embodiments.

FIG. 27 also shows the latching device 8, in particular the male part 8A, which can be switched on via a rotary lever 36 at the proximal end of the HF instrument 1. In order to ensure that the user applies sufficient closing force when the latching device 8 is switched off, an additional control device 37 in the form of a button is provided. The control device 37 is connected in an AND logic with the HF activation button 15 and/or 16 and has to be permanently actuated in order to activate HF energy.

### REFERENCE SIGNS

1 instrument
2 jaw part
3 first instrument branch
4 second instrument branch
5 blade actuating element
5A return spring of the blade actuating element
6 blade
7 HF activation element
7A inner part of the HF activation element
7B first wing of the HF activation element
70 second wing of the HF activation element
7D return spring
7E spring element
8 latching device
8A male part of the latching device
8B female part of the latching device
8C return spring of the latching device
9 first rack and pinion gear
10 input rack
11 output rack
12 pinion
13 contact element
14 activation surface
15 first HF activation button
16 second HF activation button
17 printed circuit board
18 second rack and pinion gear
19 central axis of the HF activation element
20 instrument actuating lug
21 slit
22 flat connector
23 terminal
24 first rack
25 second rack
26 pinion
27 lever
28 locking element of the HF activation element
29 plunger
30 pin
31 guiding element
32 bore
33 seal
34 sealing ring
35 hemisphere
36 rotary lever
37 control device

1.-11. (canceled)

12. A bipolar HF instrument comprising:
a jaw part, a first instrument branch and a second instrument branch for grasping tissue, which are provided and configured to move away from each other when opening and toward each other when closing;
a blade actuating element for moving a mechanical blade in the jaw part for cutting tissue; and/or
an HF activation element for activating an HF power supply for sealing tissue,
the blade actuating element and/or the HF activation element being operable from two opposite sides of the bipolar HF instrument,
the HF activation element comprising an inner part which is mounted in a rocking, sliding or pivotable manner on an axis of rotation of the HF activation element in the second instrument branch orthogonal to a proximal-distal direction,
the inner part having a side pointing in a direction proximal to the bipolar HF instrument that has a contact element with an activation surface that contacts at least a first HF activation button during actuation of the HF activation element and activates the HF power supply,
the inner part of the HF activation element comprising a first actuating wing or knob and a second actuating wing or knob, the first actuating wing or knob protruding laterally on the second instrument branch and the second actuating wing or knob protruding opposite the first actuating wing on the other side of the second instrument branch, and
the contact element comprising two adjacent cams that extend in a direction of the first HF activation button and receive the first HF activation button between the two adjacent cams without contact in a non-actuated state.

13. The bipolar HF instrument according to claim 12, wherein the HF activation element is proximally movable when performing sealing.

14. The bipolar HF instrument according to claim 12, wherein the inner part is crossbar-shaped.

15. The bipolar HF instrument according to claim 12, wherein the HF activation element is provided in an open state of the bipolar HF instrument with a locking element of the HF activation element to lock an actuation of the HF activation element.

16. The bipolar HF instrument according to claim 12, wherein the first HF activation button is arranged in a proximal direction away from the inner part of the HF activation element but facing the inner part of the HF activation element on a printed circuit board in the second instrument branch.

17. The bipolar HF instrument according to claim 12, wherein in the first instrument branch, a first rack and pinion gear is provided and arranged, in which an input rack is connected to the blade actuating element and an output rack is connected to the mechanical blade, wherein the blade actuating element is linearly movable proximally from a distal resting position for operating the mechanical blade.

18. The bipolar HF instrument according to claim 17, wherein the input rack and the blade actuating element are arranged approximately on a plane of symmetry between the first instrument branch and the second instrument branch.

19. The bipolar HF instrument according to claim 12, further comprising a latching device for achieving a latched-in state with the first instrument branch and the second instrument branch closed.

20. A bipolar HF instrument comprising:
a jaw part, a first instrument branch and a second instrument branch for grasping tissue, which are provided and configured to move away from each other when opening and toward each other when closing;
a blade actuating element for moving a mechanical blade in the jaw part for cutting tissue; and/or
an HF activation element for activating an HF power supply for sealing tissue,
the blade actuating element and/or the HF activation element being operable from two opposite sides of the bipolar HF instrument,
the HF activation element comprising a inner part mounted in a rocking, sliding or pivotable manner on an axis of rotation of the HF activation element in the second instrument branch orthogonal to a proximal-distal direction, the inner part having a side pointing in a direction proximal to the bipolar HF instrument that has a contact element with an activation surface that contacts at least a first HF activation button during actuation of the HF activation element and activates the HF power supply,
the inner part of the HF activation element comprising a first actuating wing or knob and a second actuating wing or knob, the first actuating wing or knob protruding laterally on the second instrument branch and the second actuating wing or knob protruding opposite the first actuating wing on the other side of the second instrument branch, and
a second HF activation button being provided and arranged next to the first HF activation button parallel to the inner part of the HF activation element.

21. The bipolar HF instrument according to claim 20, further comprising a latching device for achieving a latched-in state with the first instrument branch and the second instrument branch closed.

22. A bipolar HF instrument comprising:
a jaw part, a first instrument branch and a second instrument branch for grasping tissue, which are provided and configured to move away from each other when opening and toward each other when closing;
a blade actuating element for moving a mechanical blade in the jaw part for cutting tissue; and/or
an HF activation element for activating an HF power supply for sealing tissue,
the blade actuating element and/or the HF activation element being operable from two opposite sides of the bipolar HF instrument, and
the HF activation element being configured to activate the HF power supply after a first tap activation and switches off the HF power supply automatically or stops the HF power supply manually after a second tap activation of the HF activation element.

23. The bipolar HF instrument according to claim 22, further comprising a latching device for achieving a latched-in state with the first instrument branch and the second instrument branch closed.
